(19) United States
(12) Patent Application Publication
(10) Pub. No.: US 2025/0139763 A1
(43) Pub. Date: May 1, 2025

(54) MACHINE LEARNING-BASED BONE DENSITY MEASURING METHOD USING RADIOGRAPHIC IMAGE OF HIP JOINT TAKEN BY X-RAY

(71) Applicant: AIDICOME INC., Ansan-si, Gyeonggi-do (KR)

(72) Inventor: Hee Yeon KIM, Seoul (KR)

(73) Assignee: AIDICOME INC., Ansan-si, Gyeonggi-do (KR)

(21) Appl. No.: 18/280,931

(22) PCT Filed: Apr. 28, 2023
(86) PCT No.: PCT/KR2023/005877

§ 371 Date: Dec. 3, 2024

(30) Foreign Application Priority Data
Apr. 12, 2023 (KR) 10-2023-0047907

(51) Int. Cl. G06T 7/00; G06T 7/11; G06T 11/00; G06V 10/52; G06V 10/77; G06V 10/774

(52) U.S. Cl. CPC G06T 7/0012; G06T 7/11; G06T 11/006; G06V 10/7715; G06V 10/774; G06T 2207/10116; G06T 2207/20081; G06T 2207/20084; G06T 2207/30008; G06V 10/52

(57) ABSTRACT

The present invention relates to a machine learning-based bone density measuring method using a hip joint radiographic image taken by X-ray, in which a machine learning algorithm is applied to automatically generate a bone tissue image by removing soft tissue from an examinee's hip joint radiographic image taken by X-ray and to more accurately derive the examinee's bone density on the basis of image information extracted from the bone tissue image.

### TECHNICAL FIELD

The present invention relates to a machine learning-based bone density measuring method. More specifically, the present invention relates to a machine learning-based bone density measuring method using a hip joint radiographic image taken by X-ray, in which a machine learning algorithm is applied to automatically generate a bone tissue image by removing soft tissue from an examinee's hip joint radiographic image taken by X-ray and to more accurately derive the examinee's bone density on the basis of image information extracted from the bone tissue image.

### BACKGROUND ART

In general, a hip fracture refers to a state in which a hip joint that is the part of the body that connects the femur and the pelvis cracks or is broken, and is considered as one of serious injuries due to falling accidents because it is difficult to perform treatment and rehabilitation when the hip fracture occurs. Meanwhile, it is required to measure the examinee's bone density (BMD) to accurately diagnose the hip fracture or set up a treatment plan.

In general, an examinee's BMD can be measured by analyzing the amount of absorption of X-ray radiated to the bone by dual energy X-ray absorptiometry (DXA) but a DXA measuring device is expensive and occupies a large installation space. In addition, there is a method of measuring an examinee's BMD by quantitative computed tomography (QCT) but QCT requires expensive measuring equipment and has limitations in measuring BMD due to a slow scan speed.

Recently, in the medical field, research is being actively conducted on measurement of an examinee's BMD by applying a machine learning algorithm, based on a hip joint radiographic image taken by X-ray. However, the examinee's BMD cannot be accurately measured using a hip joint radiographic image taken by general X-ray due to the effect of soft tissue, which is one of noise factors, compared to when an existing DXA or QCT imaging device is used.

To solve this problem, there is a great need for a bone density measuring method, in which a machine learning algorithm is applied to automatically generate a bone tissue image by removing soft tissue from an examinee's hip joint radiographic image taken by an X-ray machine installed in most orthopedic hospitals, etc., and to more accurately derive the examinee's BMD, based on image information extracted from the bone tissue image and information about the examinee.

### DISCLOSURE

### Technical Problem

The present invention is directed to providing a machine learning-based bone density measuring method using a hip joint radiographic image taken by X-ray, in which a machine learning algorithm is applied to automatically generate a bone tissue image by removing soft tissue from an examinee's hip joint radiographic image taken by X-ray and to more accurately derive the examinee's BMD on the basis of image information extracted from the bone tissue image.

### Technical Solution

To achieve these objects, the present invention provides a machine learning-based bone density measuring method using an X-ray image, the method comprising: obtaining a hip joint radiographic image by obtaining a hip joint radiographic image, including soft tissue and bone tissue, by taking an image of an examinee's hip joint region by X-ray; generating a bone tissue image by excluding the soft tissue from the hip joint radiographic image obtained in the obtaining of the hip joint radiographic image, based on a machine learning algorithm; detecting regions of interest (ROIs) by detecting a plurality of predetermined ROIs from the bone tissue image generated in the generating of the bone tissue image; extracting image information by extracting image information corresponding to each of the ROIs detected in the detecting of the ROIs; and deriving the examinee's bone density by using the image information extracted in the extracting of the image information.

And, in the detecting of the ROIs, the plurality of predetermined ROIs may comprise the femoral neck, the femoral trochanteric region, and the femoral intertrochanteric region of the hip joint.

And the extracting of the image information may comprise extracting average grayscale values of images corresponding to the ROIs, and the deriving of the examinee's bone density comprises deriving the examinee's bone density by inputting the extracted average grayscale values to a linear regression model.

And, in the generating of the bone tissue image, the machine learning algorithm may comprise an artificial neural network algorithm to perform: an image compression process of compressing an input image into a low-dimensional image and extracting features from the compressed input image;
and an image generation process of generating a new image on the basis of the features extracted in the image compression process.

And the generating of the bone tissue image may comprise generating a bone tissue image by excluding a soft tissue image from the hip joint radiographic image obtained in the obtaining of the hip joint radiographic image, the soft tissue image being formed through the machine learning algorithm.

And, in the generating of the bone tissue image, the machine learning algorithm may build a training data set for generating a soft tissue image on the basis of the hip joint radiographic image input thereto.

And the training data set may comprise a set of an input image and an output image, the input image including synthetic images obtained by synthesizing soft tissue images and bone tissue images collected from different hip joint radiographic images taken by X-ray, and the output image including the soft tissue images.

And the input image and the soft tissue images of the output image may be collected from a soft tissue region including no bone tissue of the hip joint radiographic image.

And the bone tissue images of the input image may be collected from low-effect soft tissue images that do not include a partition region or a partition line due to the interference of the soft tissue among the hip joint radiographic images.

And the deriving of the examinee's bone density may comprise deriving bone density on the basis of at least one information among the examinee's age, height, and weight.

### Advantageous Effects

In a machine learning-based bone density measuring method using a hip joint radiographic image taken by x-ray according to the present invention, an artificial neural network algorithm, which is machine-learned to generate a soft tissue image from a hip joint radiographic image input as a training image, is applied to more accurately derive an examinee's bone density by using a bone tissue image obtained by removing soft tissue from an examinee's hip joint radiographic image.

In addition, in the machine learning-based bone density measuring method using a hip joint radiographic image taken by X-ray according to the present invention, a hip joint radiographic image taken by general X-ray is used and thus expensive measuring equipment is not required, unlike in existing bone density measuring methods, such as dual energy X-ray absorptiometry (DXA) or quantitative computed tomography (QCT). Therefore, costs required to measure an examinee's bone density can be reduced.

### DESCRIPTION OF DRAWINGS

FIG. 1 is a flowchart of a machine learning-based bone density measuring method using a hip joint radiographic image taken by X-ray according to the present invention.

FIG. 2 is an overall flowchart of a machine learning-based bone density measuring method using a hip joint radiographic image taken by X-ray according to the present invention.

FIG. 3 illustrates an overview of a machine learning algorithm used in generating a bone tissue image in a machine learning-based bone density measuring method using a hip joint radiographic image taken by X-ray, and a training data set thereof according to the present invention.

FIG. 4 illustrates each radiation absorption region on a hip joint radiographic image taken by general X-ray.

FIG. 5 illustrates a high-effect soft tissue image and a low-effect soft tissue image among hip joint radiographic images taken by X-ray.

FIG. 6 illustrates an example of a result of training a machine learning algorithm used in a machine learning-based bone density measuring method using a hip joint radiographic image taken by X-ray according to the present invention.

FIG. 7 is a graph showing a correlation between an actual bone density measured by dual-energy X-ray absorptiometry (DXA) and a bone density derived according to whether generation of a bone tissue image is included in a machine learning-based bone density measuring method using a hip joint radiographic image taken by X-ray according to the present invention.

### MODE FOR INVENTION

Hereinafter, embodiments of the present invention will be described in detail with reference to the accompanying drawings. The present invention is, however, not limited thereto and may be embodied in many different forms. Rather, the embodiments set forth herein are provided so that this disclosure will be thorough and complete, and fully convey the scope of the invention to those of ordinary skill in the art. Throughout the specification, the same reference numbers represent the same elements.

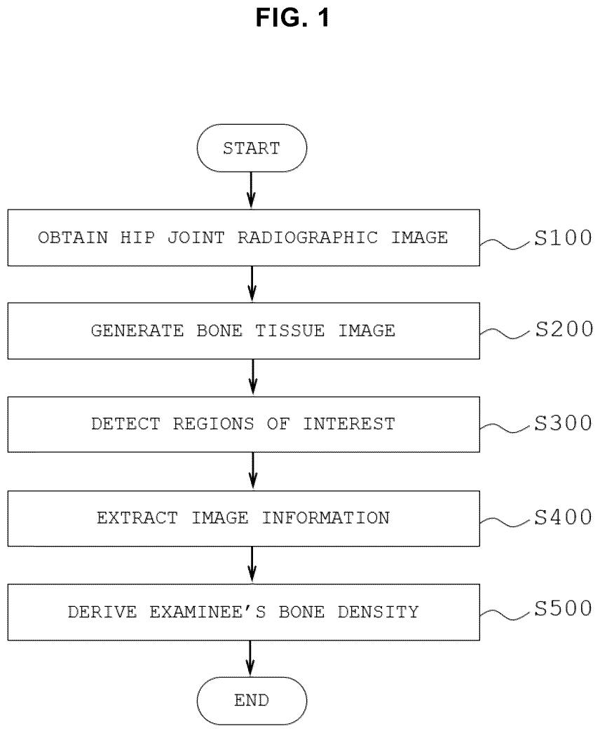

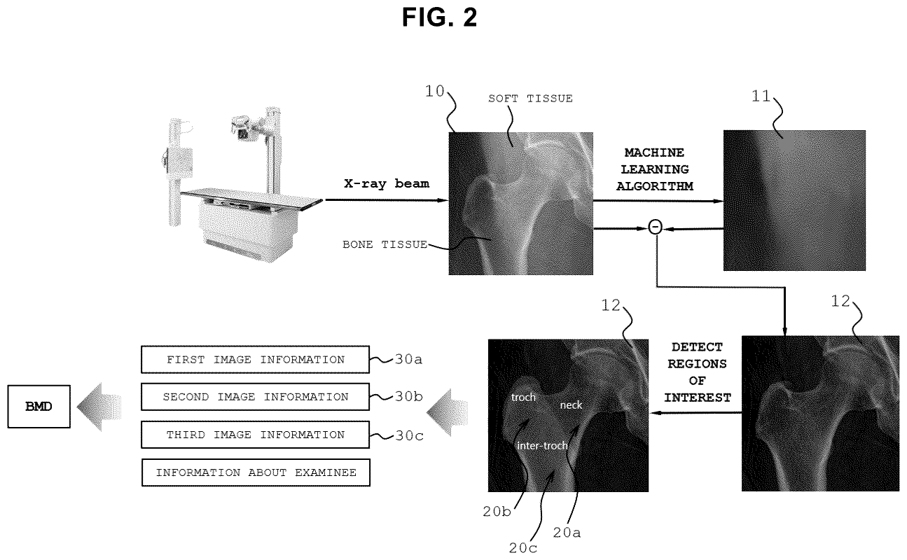

FIG. 1 is a flowchart of a machine learning-based bone density measuring method using a hip joint radiographic image taken by X-ray according to the present invention. FIG. 2 is an overall flowchart of a machine learning-based bone density measuring method using a hip joint radiographic image taken by X-ray according to the present invention.

As shown in FIGS. 1 and 2, a machine learning-based bone density measuring method according to the present invention may include: obtaining a hip joint radiographic image by taking an image of an examinee's hip joint region by X-ray to obtain the examinee's hip joint radiographic image 10 including soft tissue and bone tissue (S100); generating a bone tissue image by generating a bone tissue image 12 by removing the soft tissue from the hip joint radiographic image 10 obtained in the generating of the hip joint radiographic image, based on a machine learning algorithm (S200); detecting regions of interest (ROIs) by detecting a plurality of predetermined ROIs 20 from the bone tissue image 12 generated in the generating of the bone tissue image (S300); extracting image information by extracting image information 30 corresponding to the plurality of ROIs 20 detected in the detecting of the ROIs (S400); and deriving the examinee's bone density (BMD) from the image information 30 extracted in the extracting of the image information (S500).

In the obtaining of the hip joint radiographic image (S100), the hip joint radiographic image 10 may be obtained from image data of the examinee's hip joint region and front and rear regions of the femoral neck, which is taken by X-ray, to diagnose hip fracture. The hip joint radiographic image 10 may be obtained such that the examinee's hip joint is located at the center of the hip joint radiographic image 10 and the proximal femur looks sufficiently long.

As described above, in the present invention, by using the hip joint radiographic image 10 obtained by an X-ray imaging device, expensive measuring equipment, such as dual-energy X-ray absorptiometry (DXA) or quantitative computed tomography (QCT) which is an existing bone density measuring method, is not required and thus the hip joint radiographic image 10 of the examinee can be obtained in a relatively inexpensive and convenient manner, thereby reducing costs.

Thereafter, the generating of the bone tissue image (S200) may be performed by generating the bone tissue image 12 including only bone tissue, except for soft tissue, around the hip joint from the hip joint radiographic image 10 obtained in the obtaining of the hip joint radiographic image (S100), based on the machine learning algorithm. Here, the soft tissue should be understood to include tissues such as muscles, fasciae, skin, and fat, excluding bones or cartilage, in a hip joint region, and the bone tissue should be understood as dense connective tissue constituting bone while being surrounded by bone cells and hard calcium tissues around the bone cells.

Meanwhile, the hip joint radiographic image 10 obtained in the obtaining of the hip joint radiographic image (S100) is an image including both the soft tissue and the bone tissue, and the soft tissue included in the hip joint radiographic image 10 may be a factor that interferes with accurate prediction of the examinee's BMD. Therefore, in the generating of the bone tissue image (S200), the bone tissue image 12 including only the bone tissue of the hip joint region may be generated by removing the soft tissue from the hip joint radiographic image 10 by using the machine learning algorithm.

Specifically, in the generating of the bone tissue image (S200), the machine learning algorithm is trained to automatically generate the soft tissue image 11 on the basis of the hip joint radiographic image 10 obtained in the obtaining of the hip joint radiographic image (S100), and thus, in the generating of the bone tissue image (S200), the bone tissue image 12 may be finally generated by removing the soft tissue image 11 generated from the hip joint radiographic image 10 by the machine learning algorithm. A training method of the machine learning algorithm used in the generating of the bone tissue image (S200) will be described in detail below.

The detecting of the ROIs (S300) may be performed after the generating of the bone tissue image (S200). In the detecting of the ROIs (S300), a plurality of predetermined ROIs 20a, 20b, and 20c may be detected from the bone tissue image 12 generated in the generating of the bone tissue image (S200). The plurality of ROIs 20 detected in the detecting of the ROIs (S300) may include a first ROI 20a on the femoral neck of the hip joint, a second ROI 20b on the femoral trochanteric region of the hip joint, and a third ROI 20c on the femoral intertrochanteric region of the hip joint.

In the detecting of the ROIs (S300), the plurality of predetermined ROIs 20a, 20b, and 20c on the bone tissue image 12 may be automatically detected by applying a deep learning model such as a convolutional neural network (CNN) or be manually detected by a worker.

In the extracting of the image information (S400), the image information 30 of each of the ROIs 20a, 20b, and 20c detected in the detecting of the ROIs (S200). Here, the image information 30 extracted in the extracting of the image information (S400) may be average grayscale values of images corresponding to the ROIs 20a, 20b, and 20c.

Specifically, the images corresponding to the first ROI 20a, the second ROI 20b, and the third ROI 20c detected from the hip joint radiographic image 10 may be expressed as black-and-white images. Therefore, a plurality of pixels constituting the images corresponding to the first ROI 20a, the second ROI 20b, and the third ROI 20c detected from the hip joint radiographic image 10 may each have a grayscale value that is an integer between 0 and 255 to represent brightness information thereof. Here, among the grayscale values, ‘0’ represents black that is darkest and ‘255’ represents white that is lightest.

Therefore, in the extracting of the image information (S400), first image information 30a, second image information 30b, and third image information at 30c that are average grayscale values of the plurality of pixels of the images corresponding to the first ROI 20a, the second ROI 20b, and the third ROI 20c detected in the detecting of the ROIs (S200) may be extracted.

Thereafter, in the deriving of the examinee's BMD, the examinee's BMD may be derived from at least one piece of the image information 30 extracted from the extracting of the image information (S400).

For example, in the deriving of the examinee's BMD (S500), the examinee's BMD may be derived by inputting the average grayscale values extracted in the extracting of the image information (S400) to a linear regression model. Here, the linear regression model is a technique for modeling a linear correlation between a dependent variable and at least one independent variable, and the examinee's BMD may be estimated from existing input data by using a linear prediction function.

In the deriving of the examinee's BMD (S500), the examinee's BMD may be more accurately derived according to the examinee's individual characteristics, based on at least one piece of information, e.g., age, height, and weight, of the examinee, as well as the image information 30 extracted in the extracting of the image information (S400).

In this case, in the deriving of the examinee's BMD (S500), the linear regression model may derive the examinee's BMD by obtaining information on the basis of a correlation between information, such as age, height, and weight, of the examinee and the BMD and combining the obtained information with the average grayscale values extracted in the extracting of the image information (S400).

As described above, a main technical feature of the machine learning-based bone density measuring method using a hip joint radiographic image taken by X-ray according to the present invention is to automatically generate the bone tissue image 12 by removing the soft tissue in the generating of the bone tissue image (S200), so that an examinee's BMD may be more accurately derived on the basis of the grayscale value of the bone tissue image 12.

The machine learning algorithm used in the generating of the bone tissue image (S200) in the machine learning-based bone density measuring method using a hip joint radiographic image taken by X-ray according to the present invention will be described in more detail below.

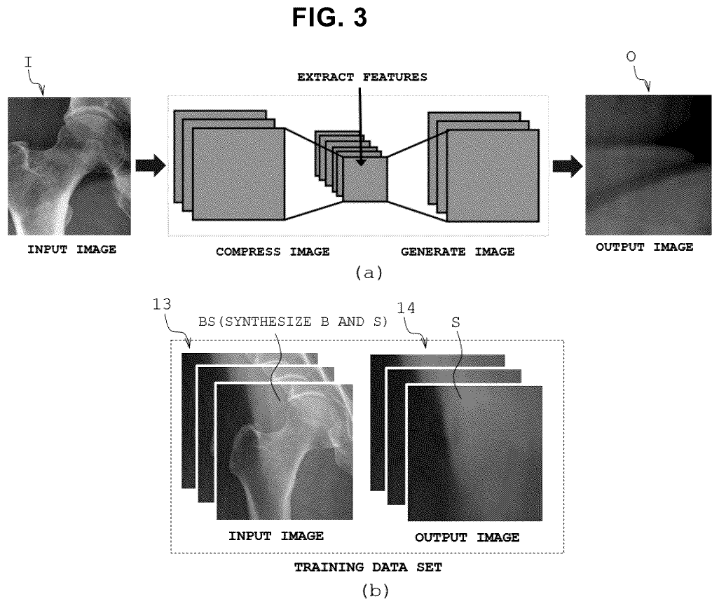

FIG. 3 illustrates an overview of a machine learning algorithm used in generating a bone tissue image in a machine learning-based bone density measuring method using a hip joint radiographic image taken by X-ray, and a training data set thereof according to the present invention.

As shown in FIG. 3A, in the generating of the bone tissue image (S200), the machine learning algorithm includes an artificial neural network algorithm trained in an unsupervised manner to perform an image compression process S210 of extracting important features from an image I input by the algorithm by compressing the image I into a low-dimensional image; and an image generation process S220 of generating a new image as similar to the input image I as possible on the basis of the features extracted in the image compression process and outputting the new image.

In the generating of the bone tissue image (S200), the image compression process S210 of the machine learning algorithm may prevent overfitting of the algorithm due to a reduction of the amount of data of the input image I, and visualize the data of the compressed input image I to accurately extract important features such as visual information, a rotation rate, a thickness, and a size.

In the generating of the bone tissue image (S200), the image generation process S220 of the machine learning algorithm may allow a user to generate a desired image from the compressed input image I on the basis of the extracted features while excluding specific features and output the desired image, when image noise is removed, image colors are changed, or texture is changed.

For example, in the generating of the bone tissue image (S200), the machine learning algorithm may be an autoencoder algorithm, and a training data set including a pair of the input image I and an output image O is required to train the machine learning algorithm.

In the generating of the bone tissue image (S200), the machine learning algorithm may be trained to perform the image compression process S210 and the image generation process S220 to generate the soft tissue image 11, including only the soft tissue, from the hip joint radiographic image 10 obtained in the obtaining of the hip joint radiographic image (S100) and output the soft tissue image 11. Accordingly, in the generating of the bone tissue image (S200), the bone tissue image 12 may be finally generated by selectively removing the soft tissue image 11 generated from the hip joint radiographic image 10 obtained in the obtaining of the hip joint radiographic image (S100) through the machine learning algorithm.

To this end, as shown in FIG. 3B, in the generating of the bone tissue image (S200), the machine learning algorithm may build a training data set for machine learning training for generating the soft tissue image 11 from the hip joint radiographic image 10.

Specifically, in the generating of the bone tissue image (S200), the machine learning algorithm may build a training data set including an input image 13 and an output image 14. The input image 13 includes images of a synthetic area BS obtained by synthesizing images of a bone tissue area B and a soft tissue area S that are collected at different locations, and the output image 14 includes images of a soft tissue area S.

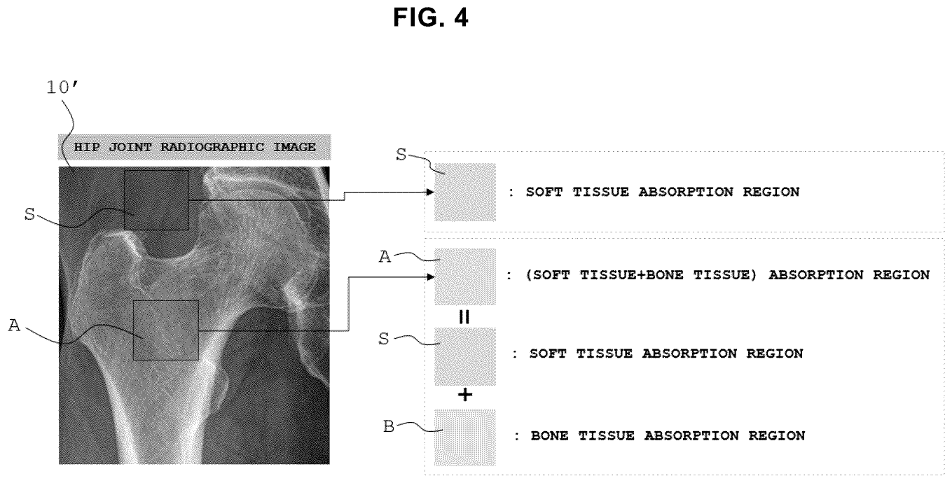

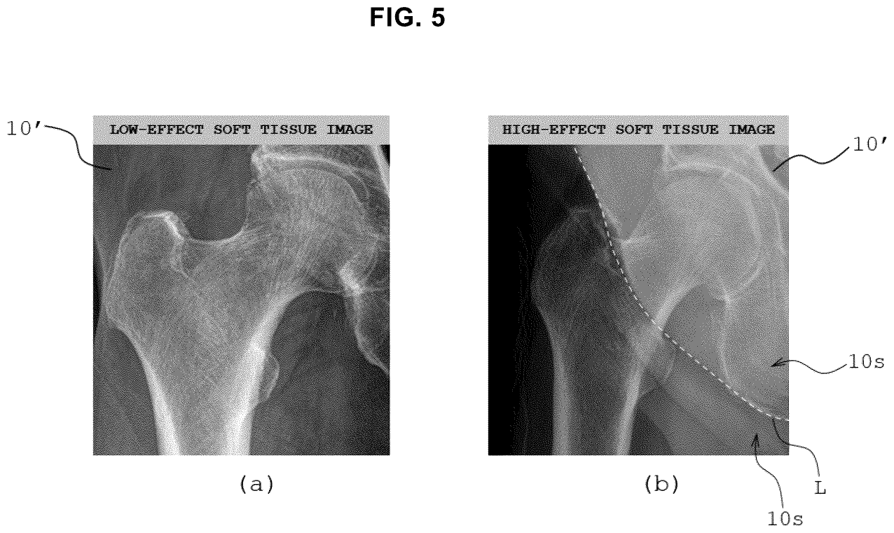

Referring to FIGS. 4 and 5, a method of collecting each of the input image 13 and the output image 14 of the training data set built in the machine learning algorithm in the generating of the bone tissue image (S200) will be described in detail below.

FIG. 4 illustrates each radiation absorption region on a hip joint radiographic image taken by general X-ray. A hip joint radiographic image 10′ shown in FIG. 4 is a training image that is input to build a training data set of a machine learning algorithm. On the hip joint radiographic image 10′ of FIG. 4, a soft tissue region S on which radiation absorption occurred only for soft tissue of a hip joint of an examinee, a bone tissue region B on which radiation absorption occurred only for bone tissue of the hip joint, and an entire region A on which radiation absorption occurred for both soft tissue and bone tissue are respectively displayed in red, blue, and purple (red+blue).

Meanwhile, the principle of DXA is based on a method of calculating the densities of the bone tissue by measuring the difference in radiation transmittance between the tissue components to be irradiated when radiation passes through the human body. Specifically, a DXA measuring device may generate different energy levels twice or more to measure BMD, generate high-energy radiation to measure a total radiation absorption rate of bone tissue and soft tissue, and generate low-energy radiation to measure a radiation absorption rate of soft tissue alone. Therefore, the DXA measuring device is capable of measuring a radiation absorption rate of only bone tissue by calculating the difference between radiation absorption rates of two different regions (an entire absorption region including bone tissue and soft tissue and a soft tissue region) by generating high-energy radiation and low-energy radiation, thereby accurately measuring an examinee's BMD.

However, the DXA measuring device is expensive equipment and is not easy to install in small hospitals. Accordingly, the present invention is directed to measuring BMD by a general X-ray device instead of the DXA measuring device. Meanwhile, BMD cannot be accurately measured using a hip joint radiographic image taken by an existing general X-ray device due to the interference or effect of soft tissue.

Therefore, as described above, according to the present invention, a machine learning algorithm in which a training data set is built may be used to repeatedly perform machine learning training for automatically generating the output image 14, including only soft tissue, from the input image 14 including both the soft tissue and bone tissue. In addition, the machine learning algorithm may generate only a bone tissue image by removing a soft tissue image, which is automatically generated through the machine learning training, from the hip joint radiographic image 10 obtained in the obtaining of the hip joint radiographic image (S100).

In the generating of the bone tissue image (S200), in order to build the training data set, the machine learning algorithm collects the soft tissue image 11 by automatically selecting the soft tissue region S, including no bone tissue, using only the hip joint radiographic image 10′ taken by X-ray, automatically collects the bone tissue image 12 from a bone tissue region B of a low-effect soft tissue image as will be described below, and combines and synthesizes these two images to form a synthetic image.

The synthetic image serving as the input image 13 is an image representing the synthetic region BS obtained by synthesizing the soft tissue region S and the bone tissue region B, and corresponds to an image to which high-energy radiation is irradiated when DXA is performed. The soft tissue image 11 serving as the output image 14 is an image representing only the soft tissue region S and corresponds to an image to which low-energy radiation is irradiated when DXA is performed.

Accordingly, in the generating of the bone tissue image (S200), a training image corresponding to a high-energy radiographic image to be used for DXA and a training image corresponding to a low-energy radiographic image of the soft tissue are included in the training data set of the machine learning algorithm. Thus, when the hip joint radiographic image 10′ is input as a training image to the machine learning algorithm, the machine learning algorithm may generate the bone tissue image 12 including only the bone tissue region B by excluding the soft tissue region S from the synthetic region BS obtained by synthesizing the soft tissue region S and the bone tissue region B, similar to DXA.

As shown in FIG. 4, the hip joint radiographic image 10′ serving as a training image includes the soft tissue region S or the synthetic region BS obtained by synthesizing the soft tissue region S and the bone tissue region B. As described above, in general, in the hip joint radiographic image 10′, either the entire absorption region A in which radiation absorption occurred for both soft tissue and bone tissue or the soft tissue region S in which radiation absorption occurred for soft tissue is provided at the location of a certain pixel of the same image.

Accordingly, conventionally, a method of building a training data set of a machine learning algorithm by collecting, using a hip joint radiographic image, an input image including the synthetic region BS obtained by synthesizing the soft tissue region S and the bone tissue region B and an output image including the soft tissue region S has not been introduced.

In contrast, according to the present invention, in order to build a training data set of a machine learning algorithm, the soft tissue region S and the bone tissue region B may be individually extracted from different hip joint radiographic images 10′ and images corresponding to the extracted two regions may be combined and synthesized to collect a synthetic image and a soft tissue image.

Specifically, the soft tissue images constituting the input image 13 and the output image 14 of the machine learning algorithm may be collected by extracting the soft tissue region S from a region of the hip joint radiographic image 10′ that includes only soft tissue. In addition, the input image 13 of the machine learning algorithm may include a synthetic image obtained by synthesizing the collected soft tissue image 11 and a bone tissue image 12 which will be described below.

However, as described above, generally, it is not possible to extract only the bone tissue region B from the hip joint radiographic image 10′ taken by X-ray, and thus, the bone tissue images constituting the input image 13 of the machine learning algorithm may be collected from a low-effect soft tissue image 10a among hip joint radiographic images.

FIG. 5 illustrates a high-effect soft tissue image and a low-effect soft tissue image among hip joint radiographic images taken by X-ray.

FIG. 5A illustrates a low-effect soft tissue image, which is less influenced by soft tissue, among hip joint radiographic images taken by X-ray. FIG. 5B illustrates a high-effect soft tissue image, which is more influenced by soft tissue, among hip joint radiographic images taken by X-ray.

Capturing a radiographic image of the body for a general treatment or diagnosis is to photograph bone tissue and thus the amount of radiation is controlled to minimize a soft tissue image. Therefore, soft tissue may be adjusted to be visible or invisible on the radiographic image by controlling the amount of radiation.

Therefore, the high-effect soft tissue image and the low-effect soft tissue image of FIG. 5 may be understood as an image on which a proportion of soft tissue that can be seen with a naked eye is high (or that can be easily seen) and low (or soft tissue that is hardly seen) respectively.

Specifically, the low-effect soft tissue image is an image of a hip joint radiographic image on which the interference or effect of the soft tissue is minimized and thus there is no partition line L or partition region 10s due to the soft tissue and bone tissue appears clearly, and the high-effect soft tissue image is an image of a hip joint radiographic image on which there is at least one partition line L or partition region 10s near the femur due to the effect of the soft tissue.

Here, the bone tissue region B of the input image 13 of the machine learning algorithm may be collected from the low-effect soft tissue image 10a among hip joint radiographic images.

Meanwhile, as a result of deriving an examinee's BMD by extracting average grayscale values of a femoral neck region, a femoral trochanteric region, and a femoral intertrochanteric region of the hip joint from thirty low-effect soft tissue images among hip joint radiographic images taken by X-ray and applying a linear regression model, a correlation coefficient between the derived examinee's BMD and the examinee's BMD measured by DXA was 0.78.

In contrast, it was confirmed that the correlation coefficient was reduced to 0.69 when the above process was repeatedly performed by additionally using 25 high-effect soft tissue images. That is, it can be seen that the effect of soft tissue on a hip joint radiographic image should be reduced to more accurately derive the examinee's BMD.

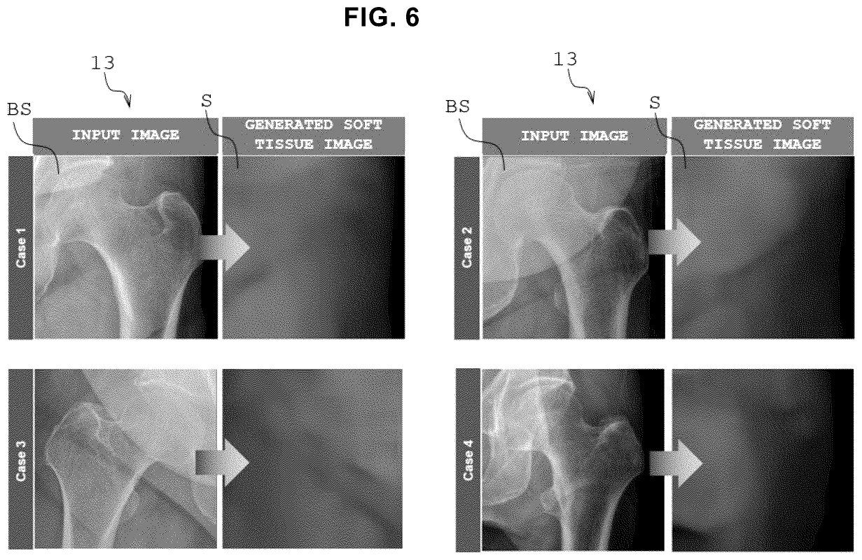

As a result, the result shown in FIG. 6 was obtained, by the machine learning algorithm in the generating of the bone tissue image (S200), collecting 2800 soft tissue regions S from hip joint radiographic images and collecting 180 bone tissue regions B from low-effect soft tissue images to build a training data set, and performing training with the training data set built in the machine learning algorithm.

FIG. 6 illustrates an example of a result of training a machine learning algorithm used in a machine learning-based bone density measuring method using a hip joint radiographic image taken by X-ray according to the present invention.

As shown in FIG. 6, it was proved by repetitive performance of a case experiment that as a result of inputting the input image 13 to the machine learning algorithm in the generating of the bone tissue image (S200), a soft tissue image including only the soft tissue region S can be generated by removing only the bone tissue region B from the synthetic region BS of the input image 13.

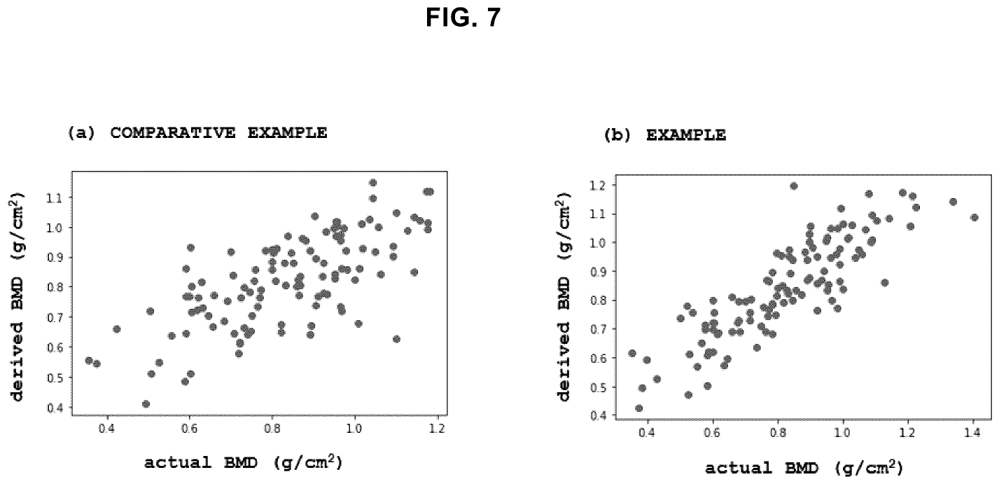

FIG. 7 is a graph showing a correlation between a BMD measured by DXA and a BMD finally derived according to whether generation of a bone tissue image is included in a machine learning-based bone density measuring method using a hip joint radiographic image taken by X-ray according to the present invention.

Specifically, image information 30 corresponding to a plurality of ROIs 20 was extracted from hip joint radiographic images 10 of 100 or more examinees and inputted to a linear regression model to derive an examinee' BMD, and a Pearson correlation coefficient between the derived examinee's BMD and an actual BMD measured by DXA was calculated. Here, as the correlation coefficient is closer to 1, a correlation between the derived examinee's BMD and the actual BMD measured by DXA is high and thus it is understood that the accuracy of the derived examinee's BMD is high.

A correlation graph of a comparative example shown in FIG. 7A reveals that a correlation coefficient between an examinee's BMD derived by DXA and the examinee's BMD, which was derived by the machine learning-based bone density measuring method using an X-ray image according to the present invention by sequentially performing the detecting of the ROIs (S300), the extracting of the image information (S400), and the deriving of the examinee's BMD (S500) by directly using the hip joint radiographic image 10 obtained in the obtaining of the hip joint radiographic image (S100) without generating the bone tissue image 12 from the hip joint radiographic image 10, was 0.715.

In addition, the correlation graph of the comparative example shown in FIG. 7A reveals that a mean absolute error (MAE) that is an error between the examinee's BMD derived by a linear regression model and an actual BMD measured by DXA was 0.109 g/cm².

A correlation graph of an example shown in FIG. 7B reveals that a correlation coefficient between an examinee's BMD measured by DXA and the examinee's BMD, which was derived by the machine learning-based bone density measuring method using an X-ray image according to the present invention by generating the bone tissue image 12 from the hip joint radiographic image 10 obtained in the obtaining of the hip joint radiographic image (S100) by applying the machine learning-based bone density measuring method described above and sequentially performing the detecting of the ROIs (S300), the extracting of the image information (S400), and the deriving of the examinee's BMD (S500), was 0.818.

In addition, the correlation graph of the example shown in FIG. 7B reveals that a MAE that is an error between the examinee's BMD derived by a linear regression model and an actual BMD measured by DXA was 0.089 g/cm².

As described above, it was confirmed that in the machine learning-based bone density measuring method using an X-ray image according to the present invention, a machine learning algorithm was used to generate the soft tissue image 11 from the hip joint radiographic image 10, so that the bone tissue image 12 may be automatically generated from the hip joint radiographic image 10 obtained in the obtaining of the hip joint radiographic image (S100) to remove or minimize noise factors in the hip joint radiographic image 10 due to soft tissue, and thus, BMD can be more accurately derived to be closest to an actual BMD measured by DXA.

While the present invention has been described above with respect to exemplary embodiments thereof, it would be understood by those of ordinary skilled in the art that various changes and modifications may be made without departing from the technical conception and scope of the present invention defined in the following claims. Thus, it is clear that all modifications are included in the technical scope of the present invention as long as they include the components as claimed in the claims of the present invention.

1. A machine learning-based bone density measuring method using an X-ray image, the method comprising:
obtaining a hip joint radiographic image by obtaining a hip joint radiographic image, including soft tissue and bone tissue, by taking an image of an examinee's hip joint region by X-ray;
generating a bone tissue image by excluding the soft tissue from the hip joint radiographic image obtained in the obtaining of the hip joint radiographic image, based on a machine learning algorithm;
detecting regions of interest (ROIs) by detecting a plurality of predetermined ROIs from the bone tissue image generated in the generating of the bone tissue image;
extracting image information by extracting image information corresponding to each of the ROIs detected in the detecting of the ROIs; and
deriving the examinee's bone density by using the image information extracted in the extracting of the image information.

2. The method of claim 1, wherein, in the detecting of the ROIs, the plurality of predetermined ROIs comprise the femoral neck, the femoral trochanteric region, and the femoral intertrochanteric region of the hip joint.

3. The method of claim 1, wherein the extracting of the image information comprises extracting average grayscale values of images corresponding to the ROIs, and
the deriving of the examinee's bone density comprises deriving the examinee's bone density by inputting the extracted average grayscale values to a linear regression model.

4. The method of claim 1, wherein, in the generating of the bone tissue image, the machine learning algorithm comprises an artificial neural network algorithm to perform:
an image compression process of compressing an input image into a low-dimensional image and extracting features from the compressed input image; and
an image generation process of generating a new image on the basis of the features extracted in the image compression process.

5. The method of claim 1, wherein the generating of the bone tissue image comprises generating a bone tissue image by excluding a soft tissue image from the hip joint radiographic image obtained in the obtaining of the hip joint radiographic image, the soft tissue image being formed through the machine learning algorithm.

6. The method of claim 5, wherein, in the generating of the bone tissue image, the machine learning algorithm builds a training data set for generating a soft tissue image on the basis of the hip joint radiographic image input thereto.

7. The method of claim 6, wherein the training data set comprises a set of an input image and an output image, the input image including synthetic images obtained by synthesizing soft tissue images and bone tissue images collected from different hip joint radiographic images taken by X-ray, and the output image including the soft tissue images.

8. The method of claim 7, wherein the input image and the soft tissue images of the output image are collected from a soft tissue region including no bone tissue of the hip joint radiographic image.

9. The method of claim 8, wherein the bone tissue images of the input image are collected from low-effect soft tissue images that do not include a partition region or a partition line due to the interference of the soft tissue among the hip joint radiographic images.

10. The method of claim 1, wherein the deriving of the examinee's bone density comprises deriving bone density on the basis of at least one information among the examinee's age, height, and weight.
